(19) United States
(12) Patent Application Publication
(10) Pub. No.: US 2011/0201905 A1
(43) Pub. Date: Aug. 18, 2011

(54) Decision support method for casualty treatment using vital sign combinations

(76) Inventor: David Spencer, San Antonio, TX (US)

(21) Appl. No.: 12/931,848

(22) Filed: Feb. 11, 2011

Related U.S. Application Data
(60) Provisional application No. 61/337,926, filed on Feb. 12, 2010.
(60) Provisional application No. 61/337,922, filed on Feb. 12, 2010.

(51) Int. Cl. A61B 5/00

(52) U.S. Cl. 600/301

(57) ABSTRACT

A device and control software and system capable of accepting patient vital sign readings from an oximeter and applying a predetermined decision algorithm that incorporates unique combinations of vital signs and will allow the care giver to continuously monitor and have continuous feedback regarding certain patient vital functions, body trauma status, and immediate treatment needs, such as the patient's likelihood of the onset of shock and the advisability of introducing blood or blood products to the patient.

This application is based upon and claims priority from U.S. Provisional applications, serial numbers 61/337,926 and 61/337,922, which are incorporated herein by reference.

### BACKGROUND OF THE INVENTION

1. Field of the Invention

Applicants' invention relates to a device for controlling the application of patient data received from an oximeter, and method for using same. More particularly, it relates to a decision support system for determining appropriate transfusion applications, which can be at or near the point of injury.

2. Background Information

For some time, medicine has focused on trying to treat patients within the “Golden Hour.” The Golden Hour is the time period after a trauma occurs in which the greatest chance that medical care will save the patient from death or serious lasting effect. Although the Golden Hour may not be exactly sixty (60) minutes, more attention is being focused on even faster treatment. Due to its experiences in Afghanistan and Iraq, the US Military has moved the state of medical practice from the “Golden Hour” to the “Platinum Ten Minutes.” In order to do this, critical care must be taken to the point of injury rather than wait for transport to a hospital. To focus on the “Platinum Ten Minutes,” the medical personal need innovative new ways to analyze and triage trauma patients.

It has been estimated that up to 90 percent of combat deaths occur on the battlefield before a casualty ever reaches a medical treatment facility. The first 10 minutes have turned out to be extremely important in helping save soldiers' lives. Likewise, civilian trauma situations, such as car wrecks, are very similar. Thus, medical first responders who have the proper training and equipment, as well as the understanding of the need for immediate evaluation and treatment, will vastly improve the chances for saving lives.

Bleeding is the leading preventable cause of death on the battlefield and in civilian trauma. 50%-75% of all trauma deaths occur before the victim reaches a hospital. In order to exploit the Platinum Ten Minutes, diagnosis and intervention must move out of the hospital and into the hands of medics allowing them to treat patients with early blood transfusions prior to reaching the hospital.

Not all bleeding victims are obviously bleeding. Internal bleeding due to trauma may be missed by a medic or paramedic. Low hemoglobin (“HgB”) indicates significant blood loss, and is known to be a powerful predictor of a bad outcome for the patient. Traditionally, HgB was measured with a laboratory machine in the hospital. However, new measurement techniques make HgB measurement available in the field.

HgB is not the only measurement available. Newly developed oximeters can provide measurements of oxygen saturation (“SpO₂”), blood perfusion, total hemoglobin (“SpHb”), oxygen content (“SpOC”), carboxyhemoglobin (“SpCO”), and methemoglobin (“SpMet”). Additional measurements may also be factors, such as sublingual partial pressure of carbon dioxide (“SlCO₂”), hemoglobin (“Hb”), pulse variability index (“PVI”), perfusion index (“PI”), near infra-red spectroscopy (“NIRS”), impedance cardiography, thromboelastography (e.g. TEG, rTEG, TEM-A, ROTEM, etc), proteomics (protein measurement), cytokines, proteins, and biomarkers. All or selected measurements of the vital signs listed above are in addition to with more traditional vital signs such as blood pressure (“BP”), heart rate (“HR”), body temperature, and respiratory rate.

One of the common effects of severe trauma and blood-loss is shock. Shock is a life-threatening condition that occurs when the body is not getting enough blood flow, and can damage multiple organs. Because of the reduced perfusion, the tissues in the body don't receive enough oxygen and nutrients to allow the cells to function, which if unchecked leads to cell death, organ damage and in turn to death. Shock can be caused by a number of different conditions, but is often associated with heavy external or internal bleeding from a serious injury. Spinal injuries can also cause shock. Shock requires immediate medical treatment and can worsen rapidly.

Given the types of trauma often found at the point of injury on the battlefield or in civilian accidents and their proclivity to inducing shock, shock is often a concern for initial responders. They are taught to look for symptoms of shock and immediately treat the patient if possible.

### SUMMARY OF THE INVENTION

The present invention is a device and control software and system capable of accepting readings from an oximeter and applying a predetermined decision algorithm that will allow the care giver to continuously monitor and have continuous feedback regarding certain patient vital functions, body trauma status, and immediate treatment needs, such as the patient's likelihood of the onset of shock and the advisability of introducing blood or blood products to the patient.

A method of the present invention is a decision support system for hemorrhage casualty treatment and resuscitation. Hemoglobin levels are also used to determine if a patient needs a blood transfusion. Usually a person's hemoglobin must be below 7-8 g/dL before a transfusion is considered. The hemoglobin concentration and readings are evaluated via a decision algorithm and the results are used to determine if a transfusion is needed, as well as how many units of packed red blood cells should be transfused. Hb measurements and changes in Hb measurements can be used in the field to better guide medics in the introduction of blood products to the patient. By determining the rate of change of hemoglobin, the present invention can indicate to the care giver whether to administer blood or blood products to the patient.

A second method of the present invention is a decision support system for using vital sign indices to predict the likelihood of the onset of shock. Additional vital signs can be read and evaluated via a decision algorithm. The results are used to determine the likelihood of shock.

The device, a hand-held vital sign monitor, would read and display the patient's vital measurements. It is anticipated that the output of the monitor could be modified by the user to display chosen measurements, as well as information about the measurements such as graphs, numerical information, trends, color warnings, sound warnings, and the like.

Upon receiving the patient's vital measurements, the hand-held vital sign monitor control software may apply one or more predetermined algorithms for determining the status of a given condition. For example, if the caregiver was worried about the patient's blood loss, then the control software would be made to evaluate the measurements and determine the need for a blood transfusion.

The results of the algorithm would be communicated by the control unit and control software to the caregiver via a graphical user interface (“GUI”) or displays on the unit. Results could be a shock predictor, transfusion indicator, or other relate and predict serious health problems such as Alzheimer's disease, asthma, breast cancer, chronic obstructive pulmonary disease, depression, diabetes, heart failure, hypertension, obesity, pleurisy, pneumothorax, sepsis, or sleep disorders.

Because there can be multiple trauma victims at a single site, it is anticipated that a single unit's control software would have the ability to recognize, track, and record measurements for multiple patients.

The invention also provides for remote monitoring of the patient. Because the measurements of vital signs and input of manually derived data, as well as the evaluations of that data by the control software and control algorithm can be transmitted over wire or wirelessly to remote treatment points, the control software and control algorithm may be utilized by remote medical personnel to monitor the patient and direct the patient's treatment.

One of the benefits of the large data sets recorded through the use of the present invention is the ability for it to use historical information from prior patients—or a first patient—regarding the measured vital signs and the patient's resulting health in order to confirm and improve upon the control software's algorithms. After measuring the first patient's vital signs, the signs are evaluated using the control algorithm and recorded on a memory device. Additionally, whether or not the first patient actually suffered the onset of a given health problem is observed, entered into the data set on the memory device, and recorded. The memory device can be internal to the vital sign monitor, or it can be an external memory device, so long as the memory device is in operable communication with the vital sign monitor. The present invention can use data informatics to help compare the recorded historical data to the current patient—or second patient—and either help predict the results of current, real-time patient data or improve the accuracy of the control algorithm in prognosticating the onset of a given health problem.

Due to the amount of data that may be measured and analyzed by the control unit and control software, large data sets will likely be developed. This is due to the number of measurements the hand-held vital sign monitors take as well as the ability to retake measurements, even to the extent of real-time measurements, in the course of monitoring a patient. The system and control software may use compression techniques for the storing and manipulating of large data sets. There are many standard compression techniques, as well as those that may arise in the future, that may be used in the present invention. It is anticipated that any of these compression techniques could be used in combination with the system or control software.

Once the patient and hand-held vital sign monitor arrive at a medical facility, the vital sign monitor can communicate the patient readings and algorithm results to other medical equipment to quickly inform medical personnel of the patient's history and status. If desired, the vital sign monitor could communicate with medical facility machines wirelessly prior to arrival in order to speed information and allow for the preparation and readiness of personnel and equipment to treat the patient upon arrival.

It is also intended that the invention control software provide for noise suppression. It is intended herein that “noise” refers not to sound, but rather to measurement artifacts that reduce the accuracy or particularity of the measurement unit's readings or the control software's calculated results. That is, as readings are made by the unit and transmitted to the controller for processing, “noise” can act to reduce the accuracy of those readings and measurements. Common mobile electronic equipment both emit and are exposed to electromagnetic noise. And, due to the intended use of the hand-held vital sign monitor or measurement unit contemplated in this invention, many other potential sources for noise exist. The noise can come from electronic sources, electromagnetic, movement of the patient or unit, chemical artifacts, biological materials, dirt, sand, mud, organic and inorganic matter, in short, any materials or interference that affect the measurements or readings taken by the vital sign monitor. Because of the host of factors and that can possibly affect the accuracy of the readings and consequently the results as determined by the control software, various noise suppression techniques may be a part of the present invention system. Noise suppression techniques for the vital sign monitors might include data fusion, filters, smoothing, sampling, interpolation techniques, Kalman filtering, the use of confidence intervals, or other techniques. The noise suppression may be applied to the raw measurements or to the results after the control algorithm has evaluated the vital signs. As used herein, noise suppression might also be referred to as signal processing. The system and control software may be used in combination with a selected noise suppression technique in order that the system or control software is able to produce more accurate results.

Patient information may be captured or documented. Life saving interventions taken need to be noted and may be by the vital sign monitor. In addition to collecting readings, generating recommendations, and recording actions taken (or not taken), all of these can be time-stamped. This record, or Casualty Care Card, may be captured by the vital sign monitor via a number of user interfaces such as a touch-screen, stylus, or audio input.

For historical reading for use, in part, for look-up tables and charts, identifying data from individual patients should be stripped from the records. Therefore the present invention contemplates the use and recording of data from all patients in order to confirm and improve the accuracy of the control software and algorithm for predicting health problems based upon the measured vital signs, as well as manually input data. Additionally, as such data is added to the history, personal and identifying information about the previous patient is stripped from the record. In this manner, the historical data serves its purpose but does not serve to disclose confidential information regarding previous patients.

### BRIEF DESCRIPTION OF THE DRAWINGS

FIG. 1. is a perspective view of one embodiment of the controller.

FIG. 2. is a schematic of the method of the present invention.

FIG. 3. is a flow-chart of the method of the present invention.

### DETAILED DESCRIPTION OF THE PREFERRED EMBODIMENT

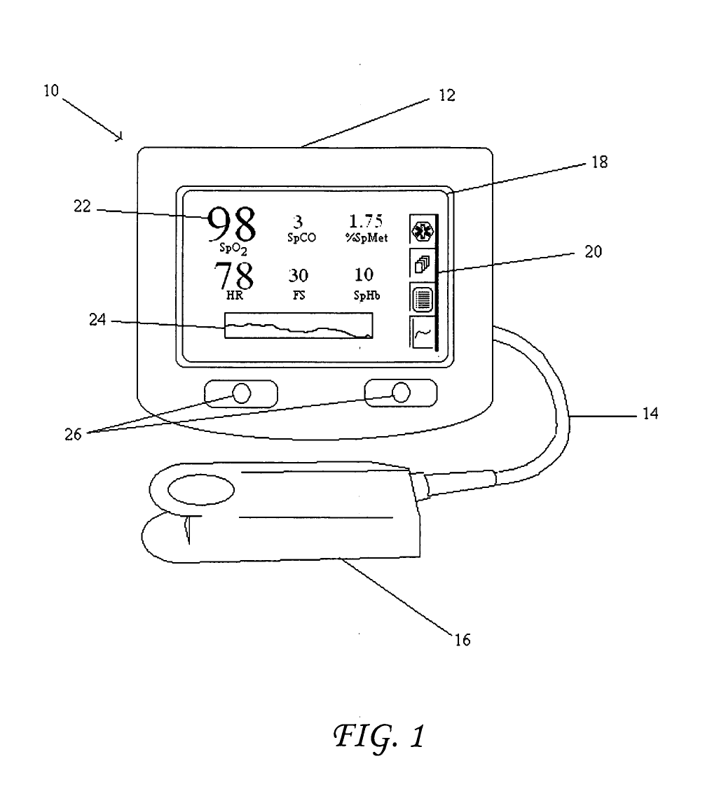

Referring to the figures, FIG. 1. illustrates a preferred embodiment of the hand-held vital sign monitor 10. The hand-held vital sign monitor 10 generally includes a meter 16, such as a finger cuff, for reading patient vital signs. The meter or oximeter 16 is in communication with the control unit 12 via a cable 14, although it is anticipated that the cable 14 could be replaced with a wireless signal between the meter 16 and the control unit 12. The meter 16 is connected appropriately to the patient in order to allow it to read the patient's vital signs. If, for example, the meter 16 is a finger cuff similar to that shown in FIG. 1, then it would be placed on the patient's finger. If necessary, multiple meters 16 could be employed in order to measure different vital signs or the vital signs of multiple patients.

The hand-held vital sign monitor 10 also generally includes a control unit 12. The control unit 12 uses innovative technology and materials to make it relatively lighter and smaller than a laboratory model in order that emergency care givers and medics can more easily carry the monitor 10. The control unit 12 also may incorporate a variety of user interfaces, these may include numerical output readings 22, graphical output readings 24, menus 20 that make the input and output of the screen 18 customizable. Additional interface mechanisms 26 may be included, such as for example, dedicated warning lights or sounds to indicate the need for an immediate transfusion. The additional interface mechanisms 26 may be either input or output in nature, and can take a variety of forms such as buttons, dials, lights, audio speakers, and the like.

The hand-held vital sign monitor 10 may incorporate anti-shock and anti-moisture technology in order to better resist environmental conditions it is likely to encounter in the field. Additionally, the control unit 12 may incorporate technology such as over-sized buttons, touch screen, stylus, and audio inputs (e.g. 18 & 20) so as to be easier to use in the field under stressful environments, such as the battlefield or in emergency situations.

The graphical user interface or screen 18 may be customized to present desired data for a given situation or concern. Thus, if blood loss is a major concern for a given patient, then the menus 20 can be manipulated such that the output data (e.g. 22 & 24) of the screen 18 is optimized for monitoring, predicting, and advising regarding blood transfusions and the administration of blood products.

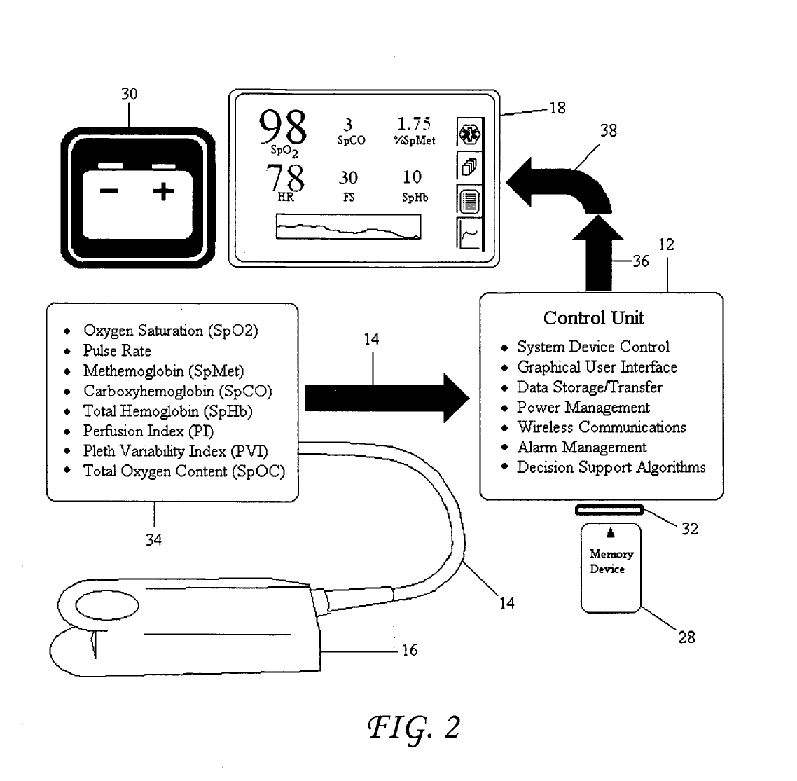

FIG. 2. is a schematic illustrating the flow of information in the method of the present invention. The vital sign monitor 10 is powered by an energy source 30, which may be a variety of power sources typically used for hand-held electronic devices such as batteries or converters from larger power sources.

The oximeter readings 34 are taken from a patient via the finger-cuff 16. The meter 16 reads a multiplicity of vital signs 34 (not all are listed in FIG. 2) including any of: HgB, SpO₂, perfusion, SpHb, SpOC, SpCO, SpMet, SlCO₂, Hb, PVI, PI, NIRS, impedance cardiography, thromboelastography (e.g. TEG, rTEG, TEM-A, ROTEM, etc), proteomics, cytokines, proteins, biomarkers, BP, HR, body temperature, and respiratory rate.

These readings 34 are communicated to the control unit 12, and the control unit 12 can display the readings via a graphical user interface or other output display 18 to the care-giver. The readings 34 are communicated to the control unit 12 via cable 14 or wirelessly. It is anticipated that the readings 34 may be communicated through many methods or devices such as audibly, visually, or electronically to other support devices.

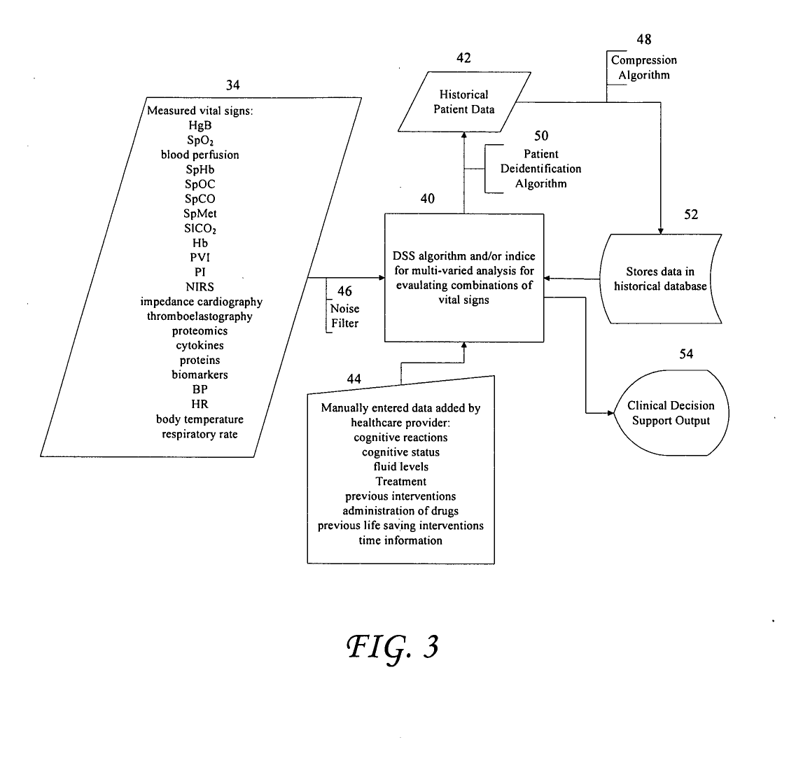

The control unit 12 employs control software, with a control algorithm (36&38 combined or shown as 40 in FIG. 3). The control unit 12 using the control software applies a predetermined control algorithm 36/38 to analyze the patient's likelihood of hemorrhaging to an extent making it necessary to administer blood products, or the vital sign indices to estimate the likelihood of onset of shock, and to provide information to the medic regarding advisable treatment for any detected hemorrhaging or predicted shock. Other critical body states may be evaluated as well. The control algorithm 36/38 uses a combination of at least two of the readings 34. The control algorithm 36/38 may include or combine two (2) or more of the measured vital signs 34, and may make use of a graphical look-up table or chart, an equation, or a comparison relationship in order to predict the onset of a serious health problem. In this instance, the table or chart would include relationships based upon historical data that prognosticates the patient's likelihood of suffering a serious health problem such as dangerous blood loss sufficient to warrant a blood transfusion or suffering the onset of shock warranting treatment for shock. The control algorithm could compare the current patient's analyzed measured vital signs to the historical analyzed vital sign measurements that led to the need of prior patients to require a blood transfusion or suffer the onset of shock the table or chart so as to prognosticate the need for said patient to require a blood transfusion or treatment for shock. Likewise, an equation or comparison relationship predicting the onset of a serious health problem could be used where the onset of the serious health problem would be resolved as a function of the combination of vital signs 34.

In alternative embodiments, the control algorithm may be split into parts, or separated even further into a multiplicity of mini control algorithms.

For example, if split into two parts, the control unit 12 may apply a predetermined evaluation algorithm to the readings 34 to analyze the patient's likelihood of hemorrhaging to an extent making it necessary to administer blood products, or the vital sign indices to estimate the likelihood of onset of shock. The control unit 12 also incorporates a predetermined decision algorithm 38. The decision algorithm 38 further evaluates the results of the evaluation algorithm 36 and provides information to the medic regarding advisable treatment for any detected hemorrhaging or predicted shock. It is anticipated that a multiplicity of smaller algorithms could take the place of the control algorithm with no, limitation as to which portion of the control algorithm each of the parts would take over.

Because the finger cuff oximeter 16 can provide readings 34 in real time or continuously, the decision algorithm 38 can take not only individual readings 34 as snap shot results, but can evaluate changes in the readings 34, the evaluation or changes in the evaluation, and combinations of the vital sign indices, as well. For example, a relatively low total hemoglobin that remains constant may not indicate that blood products are needed, while a relatively higher total hemoglobin that shows a steep change to the negative, or decrease, may indicate that the patient needs a transfusion. As a second example, a relatively low total hemoglobin that remains constant may or may not lead to shock, while the same level found in combination with other certain vital sign indice levels may indicate that onset of shock is more likely.

The control software can also incorporate manually derived data into its evaluation of the patient's previously described automatically derived vital signs or data. Manually derived data would include any data that could be added by the healthcare provider, such as the patient's cognitive reactions or status, fluid levels, treatment or previous interventions such as administration of drugs and the types, previous life saving interventions such as previous blood transfusions, time information regarding lag between the time of injury and time of monitoring or treatment.

When it is determined that the patient will likely suffer an emergency medical condition such as blood loss sufficient to warrant a blood transfusion or the introduction of blood products, or the onset of shock, the control unit 12—which is in operative communication with the screen or GUI 18—directs or controls the GUI 18 to provide communications to the user indicating that said patient will likely suffer the emergency medical condition. As a part of the output, the control unit 12 may provide specific information regarding treatment. Or, the output may be as simple as a digital—blood required/no blood required, or shock likely/shock not likely—signal, such as a “yes” or “no,” smiley face or frowny face, green light or red light, or all clear sound or warning sound. The control unit 12, through its constant monitoring of the patient's vital signs 34 can also sound an alarm if there is a catastrophic change or upon determining an emergency level has been reached. Thus, the present invention can provide users with diagnostic information, prognostic information, and care recommendations.

The control unit 12 can act as a data storage and transfer unit. There are a multiplicity of memory devices 28 that may be employed in order to store the information 34. The memory device 28 may be onboard, such as a hard-drive or RAM memory, or it may be removable as with a memory card or USB drive. Communications with any of the removable memory devices 28 would be through the appropriate memory device connection 32. This is so patient data can be maintained and later retrieved and evaluated by medical or other personnel. This may be accomplished, in various embodiments, by the controller being able to communicated or transfer data to various types of storage drives, links, and the like.

The controller may be outfitted with wireless communications capabilities—both for input and output.

In alternative embodiments, the controller may communicate with and control other medical equipment that can effect a transfusion or administration of blood products, or an administration of treatments for shock. In these embodiments, the controller evaluates the patient's hemorrhaging or likelihood of shock onset using its evaluation algorithms, determines the patient's current transfusion, transfusion rate, or treatment of shock needs using its decision algorithms, and communicates instructions to the other medical equipment so that a prescribed treatment is administered. This method is repeated in real, or near real, time so that administration is constantly monitored in order to provide the patient with the current correct treatment.

FIG. 3. is a flow-chart illustrating the flow of information in the method of the present invention. Patient vital signs 34 are measured by the vital sign monitor 10 which communicates such vital sign measurements 34 to the control algorithm 40 (or decision support system (“DSS”) algorithm). A noise filter 46 may be applied to the vital signs 34 in order to reduce measurement artifacts and electronic noise in order to increase accuracy. The health-care provider or user can manually enter data 44 in order to supplement the control algorithm 40 evaluation. The vital signs 34 and manually entered data 44 can be evaluated by the control algorithm 40. The patient vital signs 34 can be measured by the vital sign monitor 10 in real-time or near real-time. Patient vital signs 34 can be added to historical patient data 42 and stored in a historical database 52 on a memory device 28. As the vital signs 34 are added to the memory device 28, a patient deidentification algorithm 50 may be applied to the data in order to delete confidential patient information such as information that identifies the patient. Because it is anticipated that taking real time readings of a multiplicity of patients will create an extremely large data set, a compression algorithm 48 may be applied to the historical patient data 42. Historical patient data 42 from the historical database 52 can be used by the DSS algorithm 40 in order to make comparisons, update, and increase the accuracy of the DSS algorithm 40 as it evaluates new patient data—measured vital signs 34 and manually enter data 44. Once the DSS algorithm 40 evaluates the new patient data, measured vital signs 34 and manually enter data 44, the clinical decision support output 54, which can include results, diagnostics, prognostics, and recommendations as determined by the control algorithm 40, are communicated to the user via the screen 18.

While the present invention is not intended to be exclusively controlled by computer programs or algorithms, it is intended that the present invention can be implemented and controlled by computer programs or algorithms as stand-alone, over the Internet, or over other computer networks. Therefore, the present invention contemplates a series of computer algorithms and methods by which the present invention is implemented and controlled. Thus, in some of the descriptions herein, the present invention is presented partly in terms of process steps and operations of data bits within a computer memory. An algorithm is here, and generally, conceived to be a self-consistent sequence of steps leading to a desired result. These steps are those requiring physical manipulations of physical quantities. In the present invention, the operations referred to may be automated, machine operations done by a computer or similar device performed in conjunction with a human operator.

The present invention relates to the methods for operating such devices, and processing electrical, magnetic, optic, or other physical signals to generate other desired physical signals. It further relates to a computer program and the control logic contained therein. The present invention also relates to apparatus for performing these operations. The apparatus may be specially constructed for the required purposes or it may comprise a general purpose computer selectively controlled or reconfigured by a computer program stored in the memory of the computer. Further, because the present invention is intended to include a network of participants, with no geographic limitations, it is contemplated that to better implement the system of the current invention, at least part of such implementation will take place on the Internet, or other computer network. The method presented herein is not inherently related to any particular computer or other apparatus. Similarly, no particular computer programming language is required. The required structure, although not machine specific, will be apparent from the description herein. Additionally, even though a specific device or software application may, or may not, be mentioned in conjunction with a step, or algorithm, or action, it is intended that any appropriate device or software application necessary or capable of implementing that step, or algorithm, or action is anticipated herein. For example, if a step calls for the input of data, it is contemplated that any appropriate devices such as, but not limited to, various input devices, output devices, data storage devices, data transfers devices, could be used and are anticipated herein.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A method in a computer system for predicting the need for giving a patient a blood transfusion, the method comprising:
controlling a meter to measure vital signs, said vital signs being one or more chosen from the group of: hemoglobin (“HgB”), oxygen saturation (“SpO₂”), blood perfusion, total hemoglobin (“SpHb”), oxygen content (“SpOC”), carboxyhemoglobin (“SpCO”), methemoglobin (“SpMet”), sublingual partial pressure of carbon dioxide (“SlCO₂”), hemoglobin (“Hb”), pulse variability index (“PVI”), perfusion index (“PI”), near infra-red spectroscopy (“NIRS”), impedance cardiography, thromboelastography, proteomics, cytokines, proteins, biomarkers, blood pressure (“BP”), heart rate (“HR”), body temperature, and respiratory rate;
communicating said vital signs to a control unit;
analyzing two or more of said measured vital signs using a control algorithm to determine whether said patient is in need of said blood transfusion; and
when it is determined that said patient is in need of said blood transfusion, controlling a graphic user interface that is in communication with said control unit to provide communications to a user indicating that said patient is in need of said blood transfusion.

2. The method of claim 1 wherein said control algorithm incorporates at least one of a look-up table or a chart, wherein said table or chart include relationships of the historical data of said analyzed vital sign measurements that led to the need of prior patients to require a blood transfusion, and further comprising comparing said patient's said analyzed measured vital signs to said table or chart so as to prognosticate the need for said patient to require a blood transfusion.

3. The method of claim 1 wherein said control algorithm incorporates at least one of an equation or a comparison relationship in which the need of said blood transfusion is resolved as a function of two or more of said analyzed vital signs.

4. A method in a computer system for predicting the onset of shock in a patient, the method comprising:
controlling a meter to measure vital signs, said vital signs being one or more chosen from the group of: hemoglobin (“HgB”), oxygen saturation (“SpO₂”), blood perfusion, total hemoglobin (“SpHb”), oxygen content (“SpOC”), carboxyhemoglobin (“SpCO”), methemoglobin (“SpMet”), sublingual partial pressure of carbon dioxide (“SlCO₂”), hemoglobin (“Hb”), pulse variability index (“PVI”), perfusion index (“PI”), near infra-red spectroscopy (“NIRS”), impedance cardiography, thromboelastography, proteomics, cytokines, proteins, biomarkers, blood pressure (“BP”), heart rate (“HR”), body temperature, and respiratory rate;
communicating said vital signs to a control unit;
analyzing two or more of said measured vital signs using a control algorithm to determine whether said patient will likely suffer the onset of shock; and
when it is determined that said patient will likely suffer the onset of shock, controlling a graphic user interface that is in communication with said control unit to provide communications to a user indicating that said patient will likely suffer the onset of shock.

5. The method of claim 4 wherein said control algorithm incorporates at least one of a look-up table or a chart, wherein said table or chart include relationships of the historical data of said analyzed vital sign measurements that led to the need of prior patients to suffer the onset of shock, and further comprising comparing said patient's said analyzed measured vital signs to said table or chart so as to prognosticate the need for said patient to require treatment for shock.

6. The method of claim 4 wherein said control algorithm incorporates at least one of an equation or a comparison relationship in which the need of treatment for shock is resolved as a function of two or more of said analyzed vital signs.

7. A method in a computer system for predicting the onset of a health problem in a patient, the method comprising:
controlling a meter to measure vital signs, said vital signs being one or more chosen from the group of: hemoglobin (“HgB”), oxygen saturation (“SpO₂”), blood perfusion, total hemoglobin (“SpHb”), oxygen content (“SpOC”), carboxyhemoglobin (“SpCO”), methemoglobin (“SpMet”), sublingual partial pressure of carbon dioxide (“SlCO₂”), hemoglobin (“Hb”), pulse variability index (“PVI”), perfusion index (“PI”), near infra-red spectroscopy (“NIRS”), impedance cardiography, thromboelastography, proteomics, cytokines, proteins, biomarkers, blood pressure (“BP”), heart rate (“HR”), body temperature, and respiratory rate;
communicating said vital signs to a control unit;
analyzing two or more of said measured vital signs using a control algorithm to determine whether said patient will likely suffer the onset of said health problem;
wherein said health problem is one of: blood loss requiring a transfusion, shock, Alzheimer's disease, asthma, breast cancer, chronic obstructive pulmonary disease, depression, diabetes, heart failure, hypertension, obesity, pleurisy, pneumothorax, sepsis, or sleep disorder; and
when it is determined that said patient will likely suffer the onset of said health problem, controlling a graphic user interface that is in communication with said control unit to provide communications to a user indicating that said patient will likely suffer the onset of said health problem.

8. The method of claim 7 wherein said control algorithm incorporates at least one of a look-up table or a chart, wherein said table or chart include relationships of the historical data of said analyzed vital sign measurements that led to the need of prior patients to suffer the onset of said health problem, and further comprising comparing said patient's said analyzed measured vital signs to said table or chart so as to prognosticate the need for said patient to require treatment for said health problem.

9. The method of claim 7 wherein said control algorithm incorporates at least one of an equation or a comparison relationship in which the need of treatment for said health problem is resolved as a function of two or more of said analyzed vital signs.
